(19) United States
(12) Patent Application Publication
(10) Pub. No.: US 2019/0343524 A1
(43) Pub. Date: Nov. 14, 2019

(54) WOUND DRESSING COMPOSITION AND WOUND CLOSURE DEVICE MADE THEREFROM

(71) Applicant: LESLIE P. WEISER, WELLESLEY, MA (US)

(72) Inventor: LESLIE P. WEISER, WELLESLEY, MA (US)

(21) Appl. No.: 16/347,588

(22) PCT Filed: Nov. 6, 2017
(86) PCT No.: PCT/US17/60250

§ 371 Date: May 5, 2019

Related U.S. Application Data
(60) Provisional application No. 62/418,143, filed on Nov. 4, 2016.

(51) Int. Cl. A61B 17/08; A61L 24/04; A61B 90/00

(52) U.S. Cl. CPC A61B 17/085; A61B 2017/00862; A61B 90/08; A61L 24/043

(57) ABSTRACT

A wound closure device including a flexible base strip having its bottom surface coated with an adhesive material suitable for adherence to skin and constructed with bridging links spaced along the inner edge of the base strip and extending outwardly therefrom. The inner edge of the base strip is intended to be aligned adjacent to a lip of the wound being treated. Each of the bridging links has an adhesive coated section displaced from the inner edge. In the packaged or stored position, prior to engagement, the bridging links are folded over the upper surface of the adhesive strip about a hinge that is at the joint of the bridging link to the base strip. The hinge includes a partially flexible material to enable stabilizing the bridging link in an angular position. In addition, a wound dressing composite materials are disclosed for manufacturing the wound closure device.

### FIELD

The present teachings generally relate to wound dressings and wound closure devices, and more specifically to wound dressing materials and wound closure devices made therefrom.

### BACKGROUND

Among the most commonly used methods for closing wounds caused by lacerations or surgical incisions are suturing and stapling. Both of these procedures are skin invasive, which can traumatize and compromise the integrity of the wound. They increase the possibility of infection, expose the surgeon, as well as the patient, to blood-borne disease, leave behind scar tracks, and require a follow-up visit for suture or staple removal.

As is well known, a cut that invades deeply into the tissue of the skin generally requires a mechanism for drawing the sides of a wound together to promote healing and to reduce the formation of scar tissue. Surgeons have become skilled in the various techniques of suturing to minimize the resulting blemish that occurs during the healing process. These methods have always generated issues of sterilization and the very nature of suturing requires a threshold of dexterity that escapes many care providers. This is particularly true in emergency situations, which call for immediate treatment to secure the wound for transport or until such time as proper surgery is available. Suturing, even by a skilled surgeon, punctures and stresses skin tissue causing scaring. In certain situations, such as in geriatric and pediatric applications, when patient's skin is either thin and fragile, using sutures can be impractical or impossible.

It is well recognized that a sutureless wound closure would be a great benefit in many situations. Accordingly, the present disclosure provides improved sutureless wound closure devices which overcome the above issues. To enable such a sutureless wound closure device it is preferred to have a substrate material that would possess at least several desirable properties. It is preferred to have a comfortable, occlusive, breathable, hypoallergenic and transparent material as to enable observing wound healing process underneath, while ensuring that the top dermal layer to which the material is applied is not irritated and does not deteriorate over time, thus allowing the material to remain applied to skin for an extended period of time. It is also desirable to have a pressure sensitive adhesive for securing the material to skin, which has a gradually strengthening tac which is initially mild, as to allow readjusting the position of the material on skin, while curing progressively as to provide stronger subsequent skin adhesion. It is also preferred for the material to exhibit enough strength, as not to be easily tearable, but also to exhibit certain limited ability to be stretched, as to ensure drapability, while not compromising the wound closure through excessive stretching. And finally, the entire composition of the material needs to be sterilizable by both, the gamma-sterilization method and the ethylene oxide sterilization method.

### SUMMARY

In certain aspects the present teachings provide for a device for closing a wound. The device comprises all of or some of the following, an elongate, flexible base strip. The base strip has a bottom surface coated with an adhesive to adhere to one side of a wound, and a top surface opposite the bottom surface. The base also has an inner edge and an outer edge. The base strip also has a plurality of bridging links integrated with it in axially-spaced relation. The bridging links extending transversely from the inner edge of the base strip. Each bridging link can be moved about a hinge region between a first, storage position, in which the bridging links are folded over the top surface of the base strip, and a second, extended position, in which the bridging links extend outwardly from the inner edge of the base strip. Each bridging link has a first, engaging surface and a second surface opposite the engaging surface. The first engaging surface has an attachment region for attaching to another top surface of another base strip when that another base strip is placed on an opposite side of the wound and the bridging links are in the extended position. The hinge region of each bridging link comprises a partially flexible material capable of promoting stabilized orientation of the bridging link in an intermediate angular position between the first and the second positions. This device may comprise a holding tab integrated with the outer edge of the base strip and extending outwardly a predetermined distance from the outer edge. The holding tab is substantially free from adhesive and may be used for holding. The base strip of the device may be comprised of, for example, but not limited to, a loop tape of a hook-and-loop type fastener substrate. The loop tape is positioned to present loops at least on a portion of the top surface of the base strip. Complementary bridging links may comprise, for example, of a hook tape of the hook-and-loop type fastener substrate. The hook tape is oriented to present hooks in the attachment regions of the bridging links. The bridging links of the device may include a pulling tab at their outer ends. These pulling tabs are substantially free from adhesive and may be used for holding. The base strip and the bridging of the device may also be made of polyurethane or, for example, but not limited to, a breathable unidirectionally elastic substrate. The bridging links of the device may have adhesive in a locking area on the second surface. When the bridging links are in the stored position the locking area may be used to releasably secure the second surface to the top surface of the base strip. The device may have transverse indicia on the top of the bottom surfaces of the base strip. The transverse indicia may be used for guiding separating the base strip into shorter fragments. The device may also be comprised of a holding film attached to the bottom surface of the base strip at a predetermined distance from the inner edge of the base strip and extending beyond the outer edge of the base strip.

In certain aspects the present teachings provide for another device for closing a wound. The device comprises some of or all of the following, a first closure strip removably attached to a lower support sheet, and a second closure strip removably attached to the lower support sheet. The first and second closure strips are in aligned, facing relation. Each of the first and second closure strips of the device includes an elongate, flexible base strip. The base strip has a bottom surface coated with an adhesive to adhere to a first side of a wound and a top surface opposite the bottom surface. The base strip has an inner edge and an outer edge. Each of the first and second closure strips include a plurality of bridging links connected to the base strip in axially-spaced relation. The bridging links extend transversely from the inner edge. Each of the bridging links are movable about a hinge region between a first, storage position in which the bridging links are folded over the top surface of the base strip, and a second, extended position in which the bridging links extend outwardly from the inner edge. Each bridging link has a first, engaging surface and a second surface opposite the engaging surface. The first engaging surface has an attachment region for attaching to another top surface of another base strip when that another base strip is placed on an opposite side of the wound and the bridging links are in their extended positions. The hinge region of each bridging link is comprised of a partially flexible material capable of stabilizing the orientation of the bridging links in an intermediate angular position between their first and second positions. Each bridging link of the device may further be comprised of a pulling tab at its outer end. The pulling tab is substantially free from adhesive and may be used for holding. The base strip and the bridging links of the device may comprise polyurethane or, for example, but not limited to, a breathable unidirectionally elastic substrate. Each bridging link may have adhesive in a locking area on the second surface. The locking area may be used to releasably secure the bridging link to the top surface of the base strip when the bridging link is in the storage position. The device may have a transverse indicia on the top surface or the bottom surface of the first or second closure strips. The transverse indicia may be used for guiding and/or separating the first or second closure strips into shorter fragments. The device may include a first holding film removably attached to the bottom surface of the first closure strip, between the first closure strip and the support sheet. The first holding film is attached at a predetermined distance from the inner edge of the first closure strip while extending beyond the outer edge of the first closure strip. The device may also include a second holding film removably attached to the bottom surface of the second closure strip, between the second closure strip and the support sheet. The second holding film is attached at a predetermined distance from the inner edge of the second closure strip while extending beyond the outer edge of the second closure strip. When the bridging links are in their storage position, the device may include a top film removably attached to the attachment regions of the bridging links. The support sheet of the device may have an indicia line between the first and second closure strips.

### BRIEF DESCRIPTION OF THE DRAWINGS

The invention may take form in various components and arrangements of components, and in various steps and arrangements of steps. The drawings are only for purposes of illustrating preferred embodiments and are not to be construed as limiting the invention.

FIG. 1 illustrates a principal unit of wound closure device of the present teachings.

FIG. 2 illustrates an extended wound closure strip of the present teachings.

FIG. 3 shows an illustration of an extended wound closure strip of the present teachings comprised of a plurality of principal wound closure units and lines marking approximate separations between units.

FIG. 4 illustrates application of two wound closure strips of the present teachings to a wound.

FIG. 5 illustrates an exploded view of a ready-for-use wound closure device of the present teachings.

FIG. 6 illustrates an implementation of holding tabs on a wound closure strip of the present teachings.

FIG. 7A illustrates the wound dressing composite material of the present teachings.

FIG. 7B illustrates the composite wound dressing material of the present teaching comprising an additional partially flexible layer.

### DETAILED DESCRIPTION

Although the present invention will be described with reference to the embodiments shown in the figures, it should be understood that the present invention may have many alternate forms.

In the course of describing the wound closure embodiments herein, the bottom of the closing device will refer to the surface that is intended to engage the skin and the upper side or top will refer to the side of a component that is facing away from the skin after application. Directions will be indicated according to the position of the wound being treated, for example, transverse shall refer to directions across the wound. The inner edge of the closing device shall refer to the side that is intended to be adjacent to the wound lip (or edge), and the outer edge shall refer to the side of the device that is intended to be away from the wound.

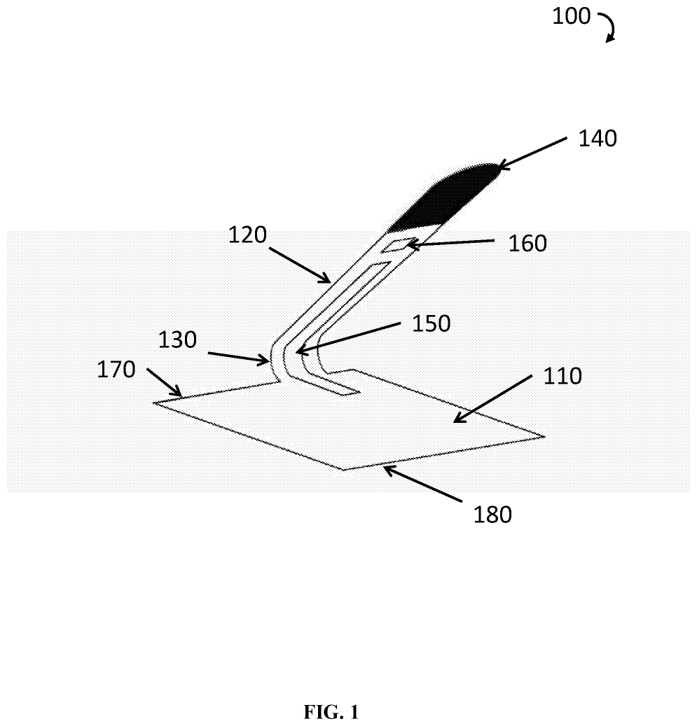

An implementation of a principal unit of the wound closure device of the present teachings is illustrated in FIG. 1. Principal unit 100 of the wound closure device comprises base 110, which has a top surface (shown in FIG. 1), a bottom surface opposite the top surface, inner edge 170, outer edge 180, and bridging link 120, which is connected to base 110 at inner edge 170 by way of hinge region 130. Bridging link 120 comprises tab 140 of appropriate size and shape as to allow for holding onto with at least two fingers. Hinge region 130 allows bridging link 120 to assume an extended (open) position, whereby bridging link 120 is substantially fully extended outwards and transversely with respect to inner edge 170; and a closed position, whereby bridging link 120 is folded backwards over the top surface of base 110. It should be clearly understood that bridging link 120 may be integrated with base 110, either by way of fusion with base 110, or by way of any other connection to base 110, i.e. chemical, thermal or mechanical bonding or incorporation. It should also be understood that the sizes and shapes of various elements shown in FIG. 1, as well as in other figures, are only illustrative and may vary, depending on specific implementations without departing from the general nature of the teachings.

The top surfaces of base 110 and bridging link 120 (all seen in FIG. 1), including hinge region 130 and tab 140, are substantially free from adhesive. Whereas the bottom surface of base 110 (not shown) as well as the bottom surface of bridging link 120 (not shown), except for tab 140, are substantially covered with adhesive which is strong enough as to ensure secure attachment of base 110 to human or animal skin while also allowing for removal of base 110 from skin if and when needed. In specific implementations it may also be desirable to leave hinge region 130 partially or completely free from adhesive, however not in the most general case contemplated herein.

It should be emphasized that one of the purposes of tab 140 is to allow for confident holding by a user of the device as to permit for easy manipulation of bridging link 120, as well as other device manipulations. Therefore, any size and shape of tab 140, while having it adhesive free as to ensure attaining holding and unhindered release thereof, is compatible with contemplated purposes of tab 140.

For storage in the closed position, adhesive may optionally cover locking area 160 on the top surface of bridging link 120, area 160 size is sufficient for securing bridging link in the folded back orientation, but may be expanded if greater tac is required. The adhesive used on locking area 160 may be the same adhesive applied elsewhere, e.g. to the bottom surface of base 110, or it may be a different, for example a lighter tac adhesive. For storage, bridging link 120 is generally folded back onto the top surface of base 110, and in case of utilizing locking area 160 the adhesive is allowed to attach to the top surface, thus securing bridging link 120 in the closed position.

Base 110 of unit 100 can be manufactured utilizing a variety of flexible or partially flexible, or unidirectionally flexible, or drapable materials. Bridging link 120, including hinge region 130 and tabs 140, may be made of the same material as base 110, in which case a single piece, including 110, 120, 130 and 140, is cut to size from a single sheet of the material selected. Alternatively, each element of the latter four elements, or any combination thereof, can be made of an individual material, or have individual additional elements. For example, in certain implementations hinge region 130 may comprise partially flexible component 150 which could promote stabilizing bridging link 120 in an intermediate angular position (illustrated in FIG. 1) between the extended (open) and the closed positions. However, the same stabilizing in the intermediate position effect can be achieved by choosing a partially flexible material for implementing any combination of the four elements that include hinge region 130. Thus, in certain implementations, base 110 may be made of a flexible material, while bridging link 120, including hinge region 130, may be made of a partially flexible material, and bridging link 120 can be attached to base 110, for example, by adhering it to the top surface of base 110, or by other equivalent methods of attachment.

The flexible material of choice for manufacturing principal unit 100 may be a non-woven, spunbond nylon material, such as ORION® or PBN-11 fabric available from Cerex Advanced Fabrics, Inc. of Cantonment, Fla. In such case utilizing partially flexible component 150 is preferred, which may comprise various partially flexible polymers, e.g. polyamides, polyesters, perforated ethylene vinyl acetate material, thin metal or nylon wires, etc.

Principal unit 100 may also be manufactured utilizing various polyurethanes, or a breathable unidirectionally elastic substrate, as for base 110, and bridging links 120, including hinge region 130 and tab 140. Utilizing a breathable unidirectionally elastic substrate allows for additional flexibility of unit 100, which may be beneficial for certain applications. This flexibility, for example, can accommodate skin expansion or shrinkage in the process of wound healing. In such partially flexible component 150 may also be optionally implemented.

For some applications principal unit 100 may be manufactured utilizing a hook-and-loop fastener substrate, such as a low-profile VELCRO® fabric available from Velcro USA, Inc. of Manchester, N.H. In this case, base 110 is manufactured of a loop tape material, having loops on the top surface of base 110 and adhesive of the loop-free bottom surface. Entire bridging link 120, including hinge region 130 and tab 140, is made of a hook tape material, having hooks on the bottom surface of bridging link 120 and thus no adhesive applied to the bridging link bottom surface. In this case utilizing partially flexible component 150 may not be necessary as hook tape itself is usually made of a partially flexible material, thus providing desirable properties in hinge region 130 of bridging link 120 as to stabilize bridging link 120 as an intermediate position. Bridging link 120 is attached to the top surface of base 110 by pressing its hooks against base 110 loops. For extra strength the attachment may be fortified by chemical bonding or thermal fusion. If necessary, smooth tab 140 on bridging link 120 can be implemented by melting down polymer hook tape hooks with local heating.

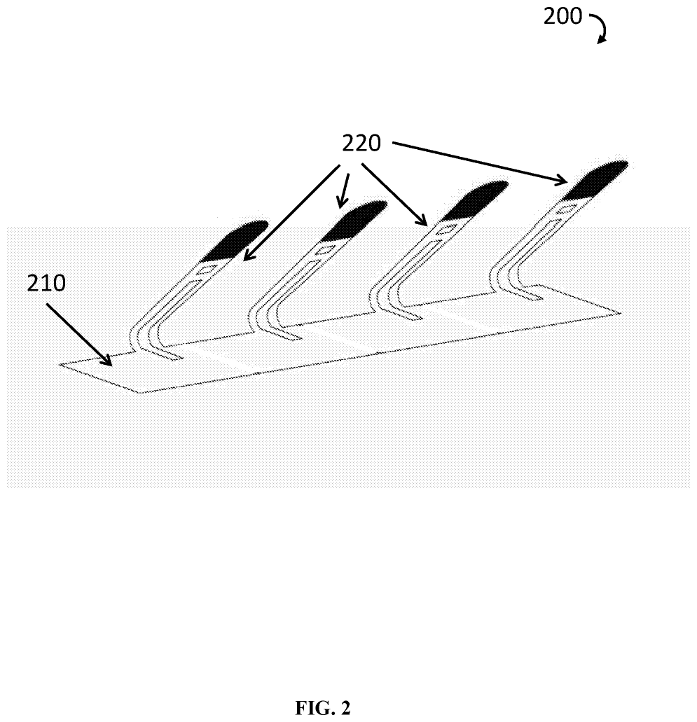

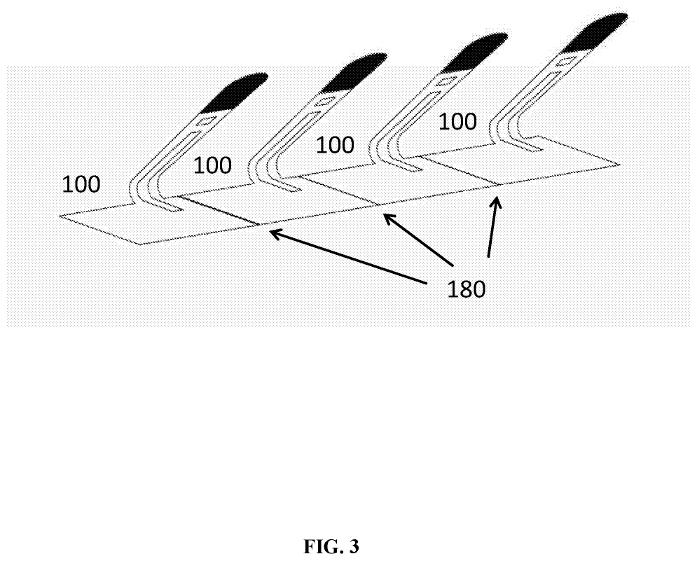

Consistent with the foregoing teachings, principal unit 100 can be realized in extended wound closure strip 200, examples of which are shown in FIG. 2 and FIG. 3. Wound closure strip 200 may be comprised of a continuous plurality of units 100 extending longitudinally, substantially along inner edge 170, thus forming extended base 210 and a plurality of bridging links 220, as shown in FIG. 2. As shown in FIG. 3, in certain implementations it may be advantages to have perforation, colored, or shaded lines 180 on extended base 210, for easy adjustments of base 210 length for particular application, or for enabling separation of wound closure strip 200 into individual units 100.

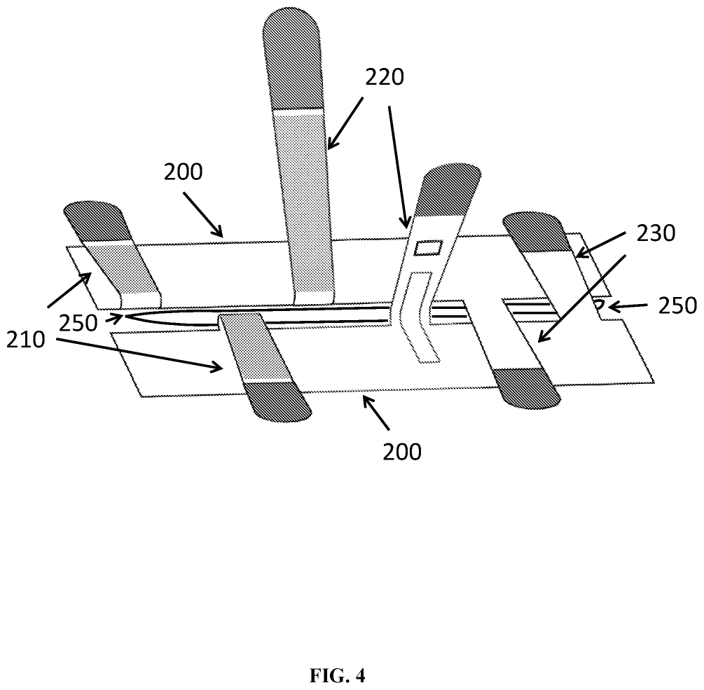

FIG. 4 shows an example of two wound closure strips 200 applied to wound 250. In the figure, one wound closure strip 200 is applied to one side of wound 250, such that the inner edge of wound closure strip 200 base is aligned with one edge of the wound substantially along that edge of the wound and at a predetermined distance from the edge of the wound. Another wound closure strip 200 is applied to the opposite side of wound 250, such that the inner edge of another wound closure strip 200 base is aligned with the opposite edge of the wound substantially along that edge of the wound and at another predetermined distance from the opposite edge of the wound. Application of the two wound closure strips 200 is done in such a manner as to have bridging links of the one wound closure strip 200 offset or facing the spaces between bridging links of the other wound closure strip 200, as illustrated in FIG. 4. Initially, the offset bridging links, facing each other, of wound closure strips 200 are in closed positions 210. For closing wound 250 the bridging links are brought into extended positions 230, when the bridging links of one wound closure strip 200 are attached to the top surface of the opposite wound closure strip 200 via the adhesive on the bottom surface of the bridging links. A complete closure of wound 250 may be achieved by bringing all bridging links into extended positions 230.

Wound closure can be readjusted, and if necessary improved, by detaching opposing bridging links from the opposite base and reattaching the bridging links while pulling the opposing bridging links into opposite direction, essentially in a “shoe string” manner. Tabs 140 on bridging links 120 (see FIG. 1) are especially useful for such detaching and reattaching of the bridging links. Before attaching bridging links for closing the wound, the bridging links can be stabilized in the intermediate position 220 (as shown in FIG. 4). This intermediate position is accomplished due to the presence of partially flexible materials in their hinge regions. Having the bridging links stabilized in intermediate positions 220 is beneficial because this allows for unhindered wound manipulation when the wound is completely or partially open for readjusting the wound closure, or for intermediate cleaning of the wound from exudates, applying medications to the wound, and/or other wound manipulations. Further, while in intermediate positions 220, the bridging links are better presented for handling by the user, especially considering that practitioners, who are most likely to use the devices of the present teachings, are routinely wearing surgical gloves. Thus, enabling stable orientation of bridging links in intermediate positions 220 also enables easier wound manipulation by a practitioner.

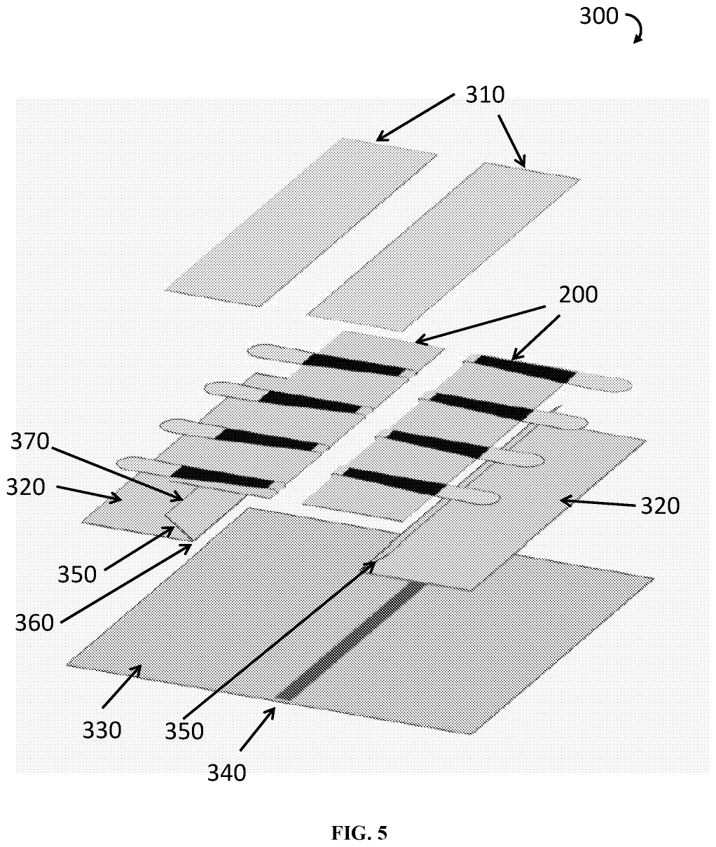

An exploded view of an example of an assembled ready-for-use wound closure device of the present teachings is illustrated in FIG. 5. Wound closure device 300 is comprised of two wound closure strips 200 assembled facing each other on support film 330. A colored or shaded line 340 on support film 330 may be used to indicate to user the wound direction. Portions of base bottom surfaces of two wound closure strips 200 are covered by holding films 320. Each holding film 320 has a folded lip 350. Each folded lip 350 is folded along lip folding line 360 and has lip outer edge 370. Two wound closure strips 200 are attached with portions of their base bottom surfaces, not covered by holding films 320, to support film 330 so as to have their inner edges facing each other and line 340 and at a predetermined distance from line 340, as illustratively shown in FIG. 5. In an assembled ready-for-use device, bridging links of each wound closure strip 200 are in closed positions, folded over backwards. Hinge regions of one wound closure strip 200 are facing into spaced between hinge regions of the opposite wound closure strip 200, as they would be when the device is applied to a wound. A portion of each wound closure strip 200 base bottom surface is covered with folded lip 350 of holding film 320, which may be clear or color coded, the remainder of holding film 320 extending beyond base outer edge as to cover at least a portion of bridging links' tabs extending beyond base outer edge. In some, but not all, implementations of the wound closure device the tabs begin at the base outer edge when bridging links are in the closed (folded backwards) position. Holding films 320 are applied to the base bottom surface of wound closure strips 200 such as to leave uncovered a portion of the base bottom surface adjacent to each base inner edge. This is achieved by placing lip outer edges 370 at a predetermined distance from base inner edges, thus leaving portions of base bottom surfaces uncovered with lips 350. The adhesive on these uncovered surfaces are used to attach wound closure strips 200 to support film 330. The top of the assembled ready-for-use wound closure device 300 is protected with clear or color coded top films 310 covering each individual wound closure strip 200 of the device by attaching to the adhesive on the bottom surfaces of the folded backwards bridging links.

Folded lips 350 may be made wide enough as to allow lip outer edges 370 to protrude a predetermined distance beyond base outer edges when holding strips 320 are attached to wound closure strips 200. The surfaces (facing bases of wound closure strips 200) of the protruding portions of folded lips 320 may be covered with adhesive for adhering to portions of bridging links protruding beyond base outer edges of wound closure strips 200 when the bridging links are in closed positions (as shown in FIG. 5). In this case having adhesive in locking area 160 of the bridging links (see FIG. 1) may not be necessary as the bridging links will be held in closed positions by adhering to the protruding portions adjacent to outer edges 370 of folded lips 350 when holding strips 320 are attached to wound closure strips 200.

Wound closure device 300 can be used as follows. Initially, one wound closure strip 200 covered with holding film 320 and top film 310 is removed from support film 330. This can be accomplished while holding onto the surfaces of films 320 and 310 to avoid adhesive covered areas of wound closure strip 200. Continuing to hold the assembly of wound closure strip 200 with films 320 and 310 after removing wound closure strip 200 from support film 330, wound closure strip 200 is applied to one side of the wound being closed with the exposed adhesive of the unprotected portion of the base adjacent to the base inner edge, leaving a predetermined distance between the edge of the wound and the base inner edge of wound closure strip 200 (as illustratively shown in FIG. 4). Because only a limited adhesive covered portion of the base of wound closure strip 200 is exposed, the rest being covered by holding film 320, it is easy to follow the edge of the wound with the base outer edge of wound closure strip 200 in case of a complex nature of the wound. If necessary, such as with an irregularly curved wound, the exposed base inner edge can be intermittently detached from skin and reattached back after a position adjustment. In certain applications, it may be desirable to remove top film 310 prior to applying the base inner edge of wound closure strip 200 to skin. In this case, only holding film 320, with protruding onto it portions of bridging links adhesive free tabs, is used for holding wound closure strip 200 while applying it to skin. After its base outer edge has been completely applied, holding film 320 is removed from under wound closure strip 200 and the remaining exposed adhesive on its base is allowed to attach to skin. After one wound closure strip 200 has been applied to one side of the wound, the other wound closure strip 200 is removed from support film 330 and applied to the opposite side of the wound in a similar fashion. During application, the two opposing wound closure strips 200 are aligned essentially as shown in FIG. 4. After both wound closure strips 200 have been securely attached to skin on the opposite sides of the wound, their bridging linked are detached from their closed positions, while holding and pulling them by their tabs (in case of adhesive covered protruding areas of folded lips 350 bridging links are released when holding film 320 is removed). The wound is closed by attaching the bridging links to the base top surfaces of the opposing wound closure strips 200, essentially in a “shoe string” manner. If it is necessary to adjust wound closure, any number of bridging links can be intermittently detached and held stably in intermediate position 220 without hindering access to the wound or neighboring bridging links, as illustrated in FIG. 4, until reattached later.

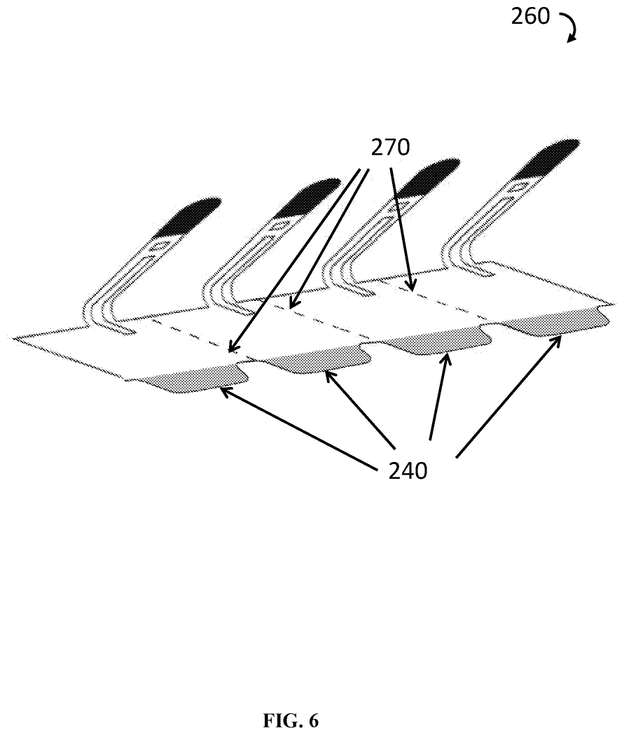

In certain implementations it may be desirable to have adhesive free holding tabs extensions on the base outer side of wound closure strips, as illustrated with color code or noncoded holding tabs 240 in FIG. 6. Holding tabs 240 may be used without holding film 320, in which case holding film 320 is not included into assembled ready-for-use wound closure device, or in combination with holding film 320. For example, holding tabs 240 may be useful for holding while separating wound closure strip 260 into shorter units along perforation lines 270, as illustrated in FIG. 6.

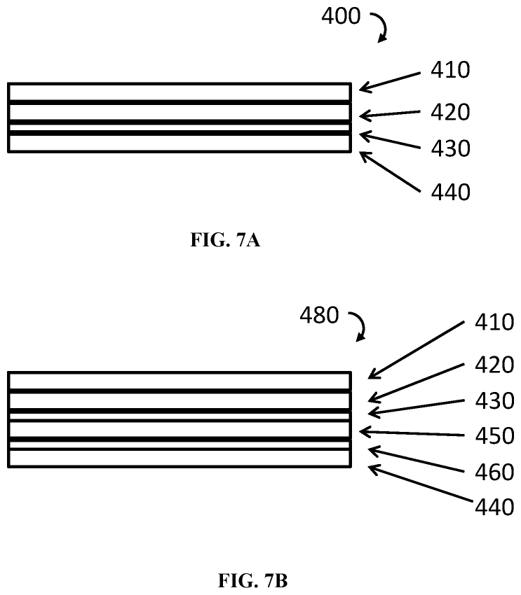

In certain implementations it is desirable to utilize the following composite wound dressing material when manufacturing the base strip of the wound closure device of the present teachings. Referring to FIG. 7A, the composite wound dressing material 400 is a multilayer sandwiched composition which contains the following layers. Layer 410 is a medical grade adhesive, for example 3M product number 1524. Layer 410 is placed over layer 420, which is a medical grade methylpenlene copolymer, for example DelStar product number PQ218. Layer 440 is a layer of medical grade polyurethane which is applied utilizing a layer of medical grade adhesive 430 over another side of layer 420. For example, MACTAC product number TM5110 can be utilized as layers 440 and 430.

In certain implementations it is desirable to utilize the following composite material when manufacturing the bridging links and the hinge region of the wound closure device of the present teachings. Referring to FIG. 7B, the composite material shown additionally contains layer 450 of medical grade polyethylene to give the resulting composite material further elastic or springy properties. Layer 450 can be inserted between layers 420 and 440, as shown in FIG. 7B, alternatively it can be inserted between layers 410 and 420 (not shown), essentially adhesive 410 is applied over layer 450 instead of layer 420. A layer 460 of medical grade adhesive is utilized to secure layer 450 in the sandwich construction of the composite material. For example, 3M product numbers 1526 or 1527 can be utilized as layers 450 and 460. 3M product number 1526 may be additionally perforated to provide for better breathability of the resulting sandwich construction.

The invention has been described with reference to the preferred embodiments. Modifications and alterations will occur to others upon reading and understanding the preceding detailed description. It is intended that the invention be construed as encompassing all such modifications and alterations insofar as they come within the scope of the appended claims or the equivalents thereof.

What is claimed is:

1. A device for closing a wound, comprising:
a flexible base strip having a bottom surface coated with an adhesive to adhere to one side of a wound, a top surface opposite the bottom surface, an inner edge and an outer edge;
a plurality of bridging links integrated with the base strip in axially-spaced relation, said bridging links extending transversely from said inner edge;
each of said bridging links movable about a hinge region between a first, storage position wherein said bridging links are folded over the top surface of the base strip and a second, extended position wherein said bridging links extend outwardly from said inner edge;
said bridging links each having a first, engaging surface and a second surface opposite the engaging surface, said first engaging surface having an attachment region for attaching to another top surface of another base strip when said another base strip is placed on an opposite side of the wound and the bridging links are in said extended position;
wherein said base strip is comprised of a composite material comprising a first layer comprising a medical grade adhesive, a second layer comprising a medical grade methylpenline copolymer, a third layer comprising another medical grade adhesive, and a forth layer comprising a medical grade polyurethane, wherein the layers are sandwitched in the following order: the first layer, followed by the second layer, followed by the third layer, followed by the forth layer; and
wherein said bridging links and the hinge region further comprise a layer of medical grade polyethylene.

2. The device of claim 1, further comprising a holding tab integrated with said outer edge of the base strip and extending outwardly a predetermined distance from said outer edge;
wherein said holding tab is substantially free from adhesive; and
wherein said holding tab may be used for holding.

3. The device of claim 1, wherein:
said another base strip comprises a loop tape of a hook-and-loop fastener substrate, said loop tape presenting loops at least on a portion of said another top surface; and
said bridging links comprise a hook take of said hook-and-loop fastener substrate, said hook tape presenting hooks in said attachment region.

4. The device for closing a wound of claim 1, wherein each of said bridging links further comprises a pulling tab at its outer end;
wherein said pulling tab is substantially free from adhesive; and
wherein said pulling tab may be used for holding.

5. The device of claim 1, wherein said base strip and said bridging links comprise polyurethane or a breathable unidirectionally elastic substrate.

6. The device of claim 1, wherein each of said bridging links further comprises adhesive in a locking area on said second surface to releasably secure said second surface to the top surface of the base strip when the bridging links are in the storage position.

7. The device of claim 1, further comprising transverse indicia on said top surface or said bottom surface of said base strip, wherein said transverse indicia may be used for guiding separating said base strip into shorter fragments.

8. The device of claim 1, further comprising a holding film attached to said bottom surface of said base strip at a predetermined distance from said inner edge of said base strip and extending beyond said outer edge of said base strip.

9. A device for closing a wound, comprising:
a first closure strip removably attached to a lower support sheet;
a second closure strip removably attached to the lower support sheet, the first and second closure strips in aligned, facing relation;
each of said first and second closure strips including an elongate, flexible base strip having a bottom surface coated with an adhesive to adhere to a first side of a wound and a top surface opposite the bottom surface, an inner edge and an outer edge;
each of said first and second closure strips including a plurality of bridging links connected the base strip in axially-spaced relation, said bridging links extending transversely from the inner edge;
each of said bridging links movable about a hinge region between a first, storage position wherein said bridging links are folded over the top surface of the base strip and a second, extended position wherein said bridging links extend outwardly from said inner edge;
said bridging links each having a first, engaging surface and a second surface opposite the engaging surface, said first engaging surface having an attachment region for attaching to another top surface of another base strip when said another base strip is placed on an opposite side of the wound and the bridging links are in said extended position; and
wherein said hinge region of each of said bridging links comprises a partially flexible material capable of promoting stabilize orientation of said bridging links in an intermediate angular position between said first and said second positions.

10. The device for closing a wound of claim 9, wherein each of said bridging links further comprises a pulling tab at its outer end;
wherein said pulling tab is substantially free from adhesive; and
wherein said pulling tab may be used for holding.

11. The device of claim 9, wherein said base strip and said bridging links comprise polyurethane or a breathable unidirectionally elastic substrate.

12. The device of claim 9, wherein each of said bridging links further comprises adhesive in a locking area on said second surface to releasably secure said second surface to the top surface of the base strip when the bridging links are in the storage position.

13. The device of claim 9, further comprising transverse indicia on said top surface or said bottom surface of said first and second closure strips, wherein said transverse indicia may be used for guiding separating said first and second closure strips into shorter fragments.

14. The device of claim 9, further comprising:
a first holding film removably attached to said bottom surface of said first closure strip, between said first closure strip and said support sheet, at a predetermined distance from said inner edge of said first closure strip and extending beyond said outer edge of said first closure strip; and
a second holding film removably attached to said bottom surface of said second closure strip, between said second closure strip and said support sheet, at a predetermined distance from said inner edge of said second closure strip and extending beyond said outer edge of said second closure strip.

15. The device of claim 14, wherein portions of the first holding film and said second holding film extending beyond said outer edge of said first and second closure strips are covered with adhesive configured to removably adhere to said bridging links when said bridging links are in said first storage position.

16. The device of claim 9, wherein said bridging links are in said storage position, and further comprising a top film removably attached to said attachment region of at least one of said bridging links.

17. The device of claim 9, wherein said support sheet further comprises an indicia line between said first and second closure strips.

18. A composite wound dressing material, said composite material comprising a first layer comprising a medical grade adhesive, a second layer comprising a medical grade methylpenline copolymer, a third layer comprising another medical grade adhesive, and a forth layer comprising a medical grade polyurethane, wherein the layers are sandwitched in the following order: the first layer, followed by the second layer, followed by the third layer, followed by the forth layer.

19. The composite dressing material of claim 18, further comprising a fifth layer comprising a medical grade polyethylene film, and a sixth layer comprising a medical grade adhesive, wherein the layers are sandwitched in the following order: the first layer, followed by the second layer, followed by the third layer, followed by the fifth layer, followed by the sixth layer, followed by the forth layer.

20. The composite material of claim 19, wherein the fifth layer is perforated.
